(19) United States
(12) Patent Application Publication
(10) Pub. No.: US 2007/0282436 A1
(43) Pub. Date: Dec. 6, 2007

(54) Stent-valve and deployment catheter for use therewith

(76) Inventor: Leonard Pinchuk, Miami, FL (US)

Correspondence Address: GORDON & JACOBSON, P.C., 60 LONG RIDGE ROAD, SUITE 407, STAMFORD, CT, 06902, US

(21) Appl. No.: 11/336,683

(22) Filed: Jan. 20, 2006

Related U.S. Application Data
(60) Provisional application No. 60/646,078, filed on Jan. 21, 2005.

(51) Int. Cl. A61F 2/24

(52) U.S. Cl. 623/2.11

(57) ABSTRACT

A stent-valve device includes a non-collapsible valve component and a stent component having a first ring connected to a second ring. The first ring has a characteristic first diameter and a valve support for supporting the valve component. The second ring is contractible and expandable between a second diameter less than a third diameter. The second diameter is less than the first diameter and the third diameter is greater than the first diameter. The first ring preferably includes a plurality of elements that extend downward to feet that project radially inward. The valve component rests on the feet for support. A seal is preferably disposed about the first ring. The valve component may be mechanical valve prosthesis, a bio-prosthesis (such as a non-collapsible porcine valve) or a polymer-based prosthesis. In another aspect of the invention, a deployment catheter is provided for effectively deploying the stent-valve device(s) described herein.

This application claims priority from provisional application 60/646,078 filed Jan. 21, 2005, which is hereby incorporated by reference herein in its entirety.

### BACKGROUND OF THE INVENTION

1. Field of the Invention

This invention relates broadly to implantable heart valves. More particularly, this invention relates to stent-valves that employ a stent for fixation of the valve.

2. State of the Art

Heart valve disease typically originates from rheumatic fever, endocarditis, and congenital birth defects. It is manifested in the form of valvular stenosis (defective opening) or insufficiency (defective closing). When symptoms become intolerable for normal lifestyle, the normal treatment procedure involves replacement with an artificial device.

According to the American Heart Association, in 1998 alone 89,000 valve replacement surgeries were performed in the United States (10,000 more than in 1996). In that same year, 18,520 people died directly from valve-related disease, while up to 38,000 deaths had valvular disease listed as a contributing factor.

Heart valve prostheses have been used successfully since 1960 and generally result in improvement in the longevity and symptomatology of patients with valvular heart disease. However, NIH's Working Group on Heart Valves reports that 10-year mortality rates still range from 40-55%, and that improvements in valve design are required to minimize thrombotic potential and structural degradation and to improve morbidity and mortality outcomes.

A large factor that contributes to the morbidity and mortality of patients undergoing heart valve replacement is the long length of time required on cardiopulmonary bypass as well as under general anesthesia. A heart valve that can be placed using minimally invasive techniques that reduces the amount of anesthesia and time on cardiopulmonary bypass will reduce the morbidity and mortality of the procedure.

Heart valve prostheses can be divided into three groups. The first group are mechanical valves, which effect unidirectional blood flow through mechanical closure of a ball in a cage or with tilting or pivoting (caged) discs. The second group are bioprosthetic valves which are flexible tri-leaflet, including (i) aortic valves harvested from pigs, (ii) valves fabricated from cow pericardial tissue, and mounted on a prosthetic stent, and (iii) valves harvested from cryo-preserved cadavers. The third group are polymer-based tri-leaflet valves.

Mechanical heart valve prostheses exhibit excellent durability, but hemolysis and thrombotic reactions are still significant disadvantages. In order to decrease the risk of thrombotic complications patients require permanent anticoagulant therapy. Thromboembolism, tissue overgrowth, red cell destruction and endothelial damage have been implicated with the fluid dynamics associated with the various prosthetic heart valves.

Bioprostheses have advantages in hemodynamic properties in that they produce the central flow characteristic to natural valves. Unfortunately, the tissue bioprostheses clinically used at present also have major disadvantages, such as relatively large pressure gradients compared to some of the mechanical valves (especially in the smaller sizes), jet-like flow through the leaflets, material fatigue and wear of valve leaflets, and calcification of valve leaflets (Chandran et al., 1989).

Polymer-based tri-leaflet valves are fabricated from biochemically inert synthetic polymers. The intent of these valves is to overcome the problem of material fatigue while maintaining the natural valve flow and functional characteristics. Clinical and commercial success of these valves has not yet been attained mainly because of material degradation and design limitations.

### SUMMARY OF THE INVENTION

It is therefore an object of the invention to provide a heart valve device that provides for natural valve flow and functional characteristics with minimal material degradation.

It is another object of the invention to provide such a heart valve device that is efficiently and effectively fixated within the heart.

It is a further object of the invention to provide such a heart valve device with minimal and hemolysis and thrombotic reactions.

In accord with these objects, a stent-valve device is provided that includes a non-collapsible valve component and a stent component having a first ring connected to a second ring. The first ring has a characteristic first diameter and a valve support for supporting the valve component. The second ring is contractible and expandable between a second diameter less than a third diameter. The second diameter is less than the first diameter and the third diameter is greater than the first diameter. The stent component is preferably realized from at least one shape memory metal. The non-collapsible valve component preferably comprises a substantially rigid annular base and a plurality of flexible leaflets that extend from its base. The non-collapsible valve component may be a mechanical valve prosthesis, a bio-prosthesis (such as a non-collapsible porcine valve) or a polymer-based prosthesis.

According to one embodiment, the first ring of the stent component includes a plurality of elements that extend downward to feet that project radially inward. The valve component rests on the feet for support. A seal is preferably disposed about the first ring.

According to another embodiment, a plurality of suspension elements connect the first ring to the second ring to thereby allow the first ring to hang below the second ring in use.

According to a preferred embodiment, the second ring comprises a band of hexagonal elements having upper and lower apices that extend radially outward in a manner that fixates the stent-valve device in place against an inner wall of a blood vessel.

In another aspect of the invention, a deployment catheter is provided for effectively deploying the stent-valve device(s) described herein. The deployment catheter includes a first housing that is adapted to store the second ring in its contracted state, and a first body member adapted to move the first housing axially to deploy the second ring from the first housing. A restrictor member is operably disposed adjacent the second ring. The restrictor member is adapted to limit axial movement of the second ring while the first body member is moved axially to deploy the second ring. A second body member, preferably concentric over the first body member, is manipulated to effectuate axial movement of the first housing relative to the restrictor member.

According to one embodiment, the deployment catheter includes a second housing that is adapted to extend through the valve component (e.g., through the flexible leaflets and base of the valve component). The second housing is retracted therefrom after deploying the second ring. Preferably, a third body member is provided, which is concentric over the first and second body members, to allow for axial movement of the second housing relative to the restrictor member and the first housing.

Additional objects and advantages of the invention will become apparent to those skilled in the art upon reference to the detailed description taken in conjunction with the provided figures.

### BRIEF DESCRIPTION OF THE DRAWINGS

FIG. 1 is an isometric view of the stent component of an exemplary stent-valve device in accordance with the present invention.

FIG. 2 is an isometric view of valve component of an exemplary stent-valve device in accordance with the present invention.

FIG. 3 is an isometric view of an exemplary stent-valve device in accordance with the present invention, wherein the valve component of FIG. 2 is placed within the stent component of FIG. 1.

FIG. 4 illustrates an exemplary stent valve device with a seal operably disposed around the lower securing ring with the upper fixation ring compressed radially inward into a compressed state which is suitable for loading into the upper nose of a deployment catheter as shown in FIGS. 5-10.

FIGS. 5-9 are cross section views of the operations of an exemplary deployment catheter for deploying and fixating the stent-valve device of FIG. 3 to its intended deployment site where it is secured to the inner wall of a blood vessel.

FIG. 10 is an isometric view of the deployment catheter of FIGS. 5-9.

FIG. 11 is a pictorial illustration of the heart showing the stent-valve device of FIG. 3 positioned in the ascending aorta upstream from left ventricle.

FIGS. 12-14 are cross section views of the operations of another deployment catheter for deploying and fixating the stent-valve device of FIG. 3 to its intended deployment site where it is secured to the inner wall of a blood vessel.

FIG. 15 is an isometric view of an alternate stent component for a stent-valve device in accordance with the present invention.

FIG. 16 is an isometric view of a stent-valve device in accordance with the present invention, wherein the valve component of FIG. 2 is placed within the stent component of FIG. 15 with a seal operably disposed around the suspenders of the stent and the valve component supported there.

### DETAILED DESCRIPTION

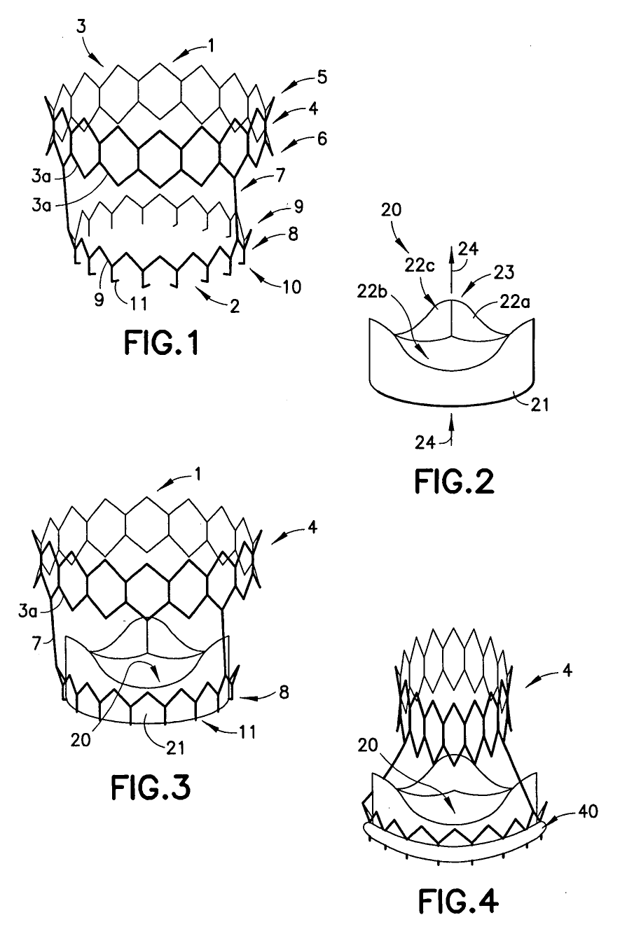

Turning now to FIG. 1, there is shown the stent component 1 of a stent-valve in accordance with the present invention. The stent 1 is typically made from a laser machined shape memory metal such as nitinol or Elgiloy or any other medical grade metal suitable for stents, stent-grafts and the like. Further, the stent component can be made using wire forms with and without welding. The stent 1 consists of a proximal end 2 opposite a distal end 3. The distal end 3 contains a band of hexagonal shaped elements with adjacent elements sharing a common side. This band of hexagonal elements is herein called a fixation ring 4. The fixation ring 4 can also be comprised of diamond shaped or zig-zag shaped elements, etc. Each hexagonal element 3a is formed in a geometry such that both the upper apices 5 and the lower apices 6 extend radially outward from the central portion of the fixation ring 4 as best shown in FIGS. 1 and 3. The purpose of the angle of the apices 5 and 6, as will later be demonstrated, is to contact the inner wall of a blood vessel in order to prevent the stent from moving distally (or proximally) in the blood vessel; in other words, such apices fixates the stent in place against the inner wall of the blood vessel.

A plurality (preferably, at least three) suspenders or connectors 7 hang from the fixation ring 4 and attach the fixation ring 4 to a lower securing ring 8. The securing ring 8 preferably comprises a band of zig-zag elements 9 (although this ring 8 can also include diamond shaped or hexagonal shaped elements, etc.). The lower part of the securing ring 8 is comprised of elements 10 that project generally downward to feet 11 that project radially inward. The securing ring 8 is suspended in place by the fixation ring 4.

FIG. 2 illustrates an exemplary non-collapsible prosthetic heart valve 20 for use in conjunction with the present invention. The valve 20 includes a substantially rigid annular base 21 with three flexible leaflets 22a, 22b, 22c attached along its upper surface 23. The base 21 and leaflets 22a, 22b, 22c may be formed from a biochemically inert polymeric material. Alternatively, the rigid base may be formed from a metal, such as titanium, stainless steel, nitonol, etc. It will be appreciated by those skilled in the art that fluid flowing in the direction of arrow 24 will displace the leaflet 22a, 22b, 22c axially and move through a central gap formed by the axial displacement of the leaflets 22a, 22b, 22c; while fluid traveling in the opposite direction of arrow 24 will cause the leaflets 22a, 22b, 22c to close by opposing each other and thus block the flow of fluid in this opposite direction. Any other non-collapsible prosthetic heart valve may be used, including, but not limited to, mechanical valves (e.g., tilting disk), non-collapsible bioprosthetic valves and other non-collapsible polymer-based prosthetic valves.

FIG. 3 shows the valve 20 placed in the stent 1 with the base 21 of the valve resting on the feet 11 of the stent. It will be appreciated by those skilled in the art that the valve 20 can be sutured, glued to, mechanically attached, force fit, locked into or otherwise rigidly attached to the securing ring 8 of the stent 1. It can further be appreciated that the securing ring 8 may be heat treated at a very small diameter and expanded such that valve 20 fits into the securing ring stent such that inward forces of the expanded securing ring hold the valve 20 in place. It should be noted that this is the reverse of a typical stent design that relies on outward forces to hold it in place. It can also be appreciated by those skilled in the art that the feet 11 can be designed as a harness or the like to capture the valve 20 which will enable easy assembly of the stent-valve in the operating room.

As shown in FIG. 4, a seal 40 is preferably disposed around the securing ring 8. The seal may be an annulus of foam, a multiplicity of strands, a rolled sewing cuff, or the like. The seal 40 prevents blood from leaking around the device once it is fixated. In addition, the seal 40 can be made porous to allow tissue ingrowth and facilitate permanent fixation of the device. Further, for certain applications, such as for aortic valve replacement as discussed below, the seal 40 can also take the form of an annular wedge such that a wide potion of the wedge remains in the ventricle, while the remaining portion of the wedge lies in the aorta, much like a cork in a bottle.

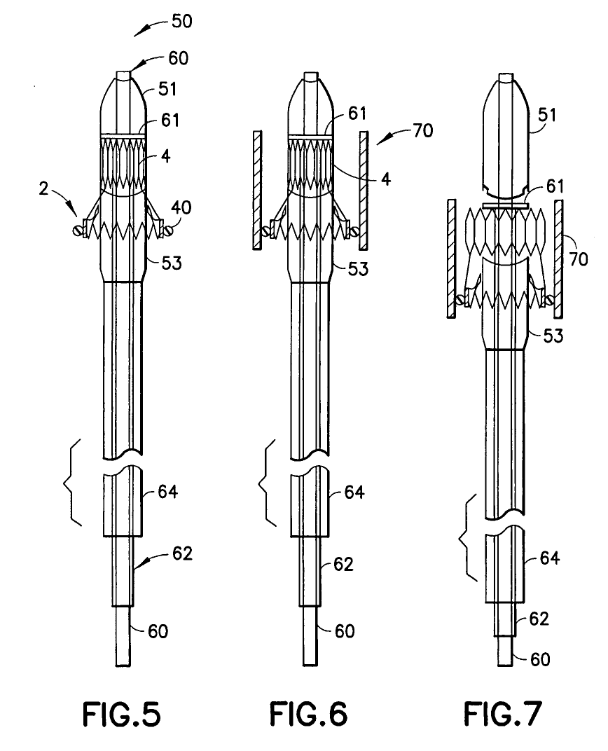

In another aspect of the present invention, the stent valve device described above is loaded into and deployed from a deployment catheter as shown in FIGS. 4-10. After the valve 20 is secured in place to the securing ring 8 and the seal 40 disposed around the securing ring 8, the fixation ring 4 is compressed radially inwards as shown in FIG. 4. A catheter 50 is provided with an upper nose cone 51 rigidly secured to an inner-body 60 as shown in FIG. 5. The inner-body 60 can be hollow to accommodate a guide wire, endoscope, fiber optics, fluid passage way, and the like. The inner-body 60 extends the entire length of the catheter where it can terminate with a hub with a luer or the like (not shown). The nose cone 51 holds the fixation ring 4 in its compressed state while the catheter is guided through the vasculature to the deployment site.

A restrictor 61 is rigidly secured to a mid-body 62. The mid-body 62 is concentric over the inner-body 60 and can be attached to a grip or the like (not shown) to enable holding in place during deployment. The restrictor 61 is disposed distally adjacent the fixation ring 4 and prevents the fixation ring from moving distally when the nose cone 51 is moved forward to enable deployment of the stent-valve device.

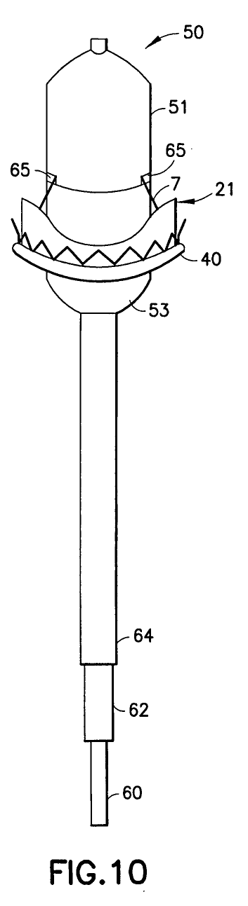

The deployment catheter 50 also includes a second inverse or lower cone 53 securely attached to an outer-body 64. The outer-body 64 is concentric over the mid-body 62 and can be attached to a grip or the like (not shown) to enable holding in place during deployment. The second cone 53 is inserted through the valve 20 (e.g., through the flexible leaflets and base the valve) where it nests or otherwise mates concentrically with the upper nose cone 51 as best shown in FIGS. 5 and 10.

The proximal end of the upper nose cone 51 includes cutouts 65 through which pass the suspenders 7 of the stent as the stent is fixation ring 4 is held in its compressed state under the upper nose cone 51 as best shown in FIGS. 5 and 10.

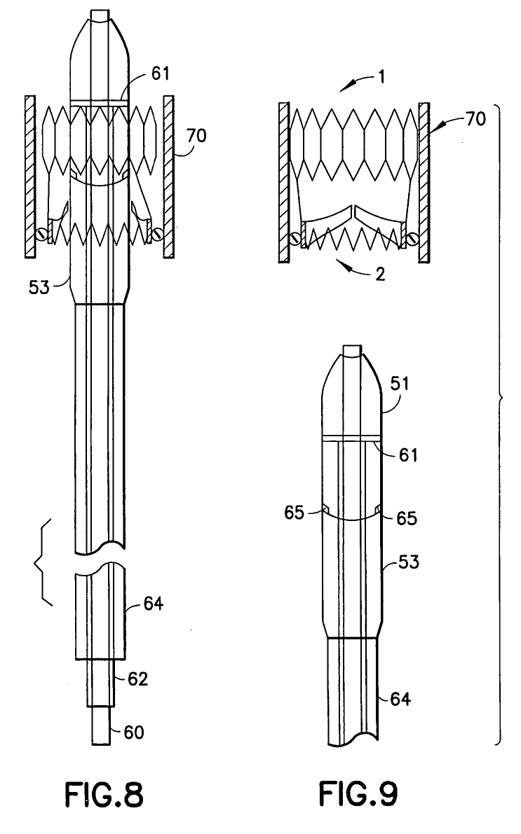

The stent-valve is deployed as shown in FIGS. 6-9. The catheter 50 (and the stent-valve housed therein as shown in FIGS. 5 and 10) is introduced into the deployment area preferably by an intercostal penetration methodology. The catheter is then positioned in place at the deployment site (FIG. 6). While the restrictor 61 is held in place by securing the mid-body 62, the upper nose cone 51 is advanced forward thereby allowing the fixation ring 4 to deploy (FIG. 7). The outward radial force produced by the fixation ring 4 combined with the angled orientation of the apices of the fixation ring 4 securely attach the fixation ring 4 to the vessel wall 70. The suspenders 7 and securing ring 8 with feet 11 hold the valve 20 in place and the seal 40 prevents fluid from flowing around the valve 20. After the fixation ring 4 is deployed, the entire catheter assembly is retracted through the valve 20 by pulling the bodies 60, 62, 64 rearward (FIGS. 8 and 9) and out of the body.

The lower cone 53 is shaped to mate with the upper nose cone and thereby protect the leaflets of the valve 20 from damage when the assembly is retracted back through the leaflets after deployment. FIG. 9 shows the stent-valve assembly deployed and secured to the vessel wall 70 at the deployment site. FIG. 10 illustrates the stent-valve assembly loaded into the deployment catheter 50 prior to introduction into the body.

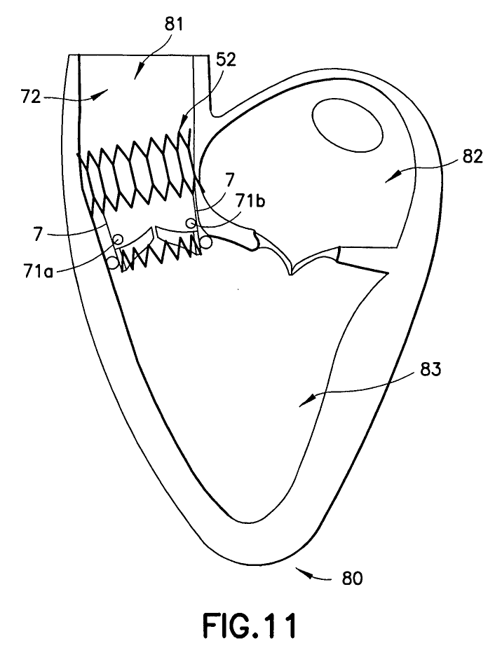

FIG. 11 illustrates the deployment and fixation of the stent-valve assembly of the present invention in the ascending aorta 72. It can be located at or near the original location of a removed aortic valve or it can be inserted through an old aortic valve where it essentially pushes the leaflets of the old aortic valve aside. It is placed in the ascending aorta 72 just distal to the left ventricle 83 with the upper fixation ring 4 located distal to the coronary arteries 71a, 71b and the lower securing ring 8 placed proximal to the coronary arteries 71a, 71b and above the ventricle. The suspenders 7 of the stent are rotated/located so as not to interfere with blood flow to the coronary arteries 71a, 71b. The deployment catheter 50 is inserted below the deployment site through the wall of the left ventricle 83 by cutting a slit in the left ventricle at site 80 which is thereafter repaired. Alternate entrance sites within the left ventricle 83 may be used. The left atrium 82 and left ventricle 83 are shown as landmarks within the heart for simplicity of description.

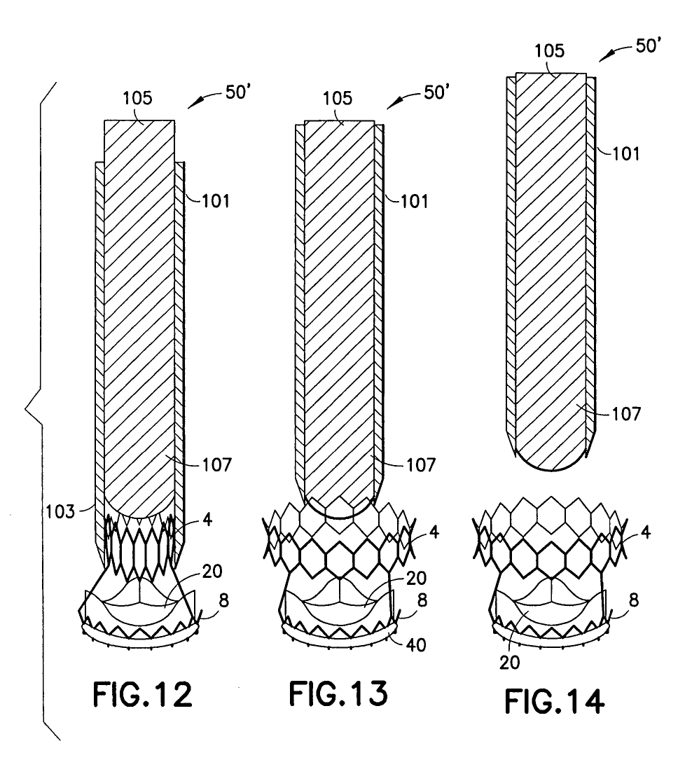

Alternatively, the stent-valve assembly can be deployed from above the deployment site (e.g., from the aorta where a slit can be made, for example, at site 81 as shown in FIG. 11). In this alternative embodiment, the fixation ring 4 is disposed proximal relative to the securing ring 8. A deployment catheter 50′ as shown in FIGS. 12-14 can be used to deploy the stent-valve at the intended deployment site. The catheter 50′ includes an outer cannula 101 whose distal end 103 holds the fixation ring 4 in its compressed state as shown in FIG. 12. An inner push rod 105 is disposed within the outer cannula 101 with its distal end 107 disposed adjacent the fixation ring 4. The inner push rod 105 can be hollow to accommodate a guide wire, endoscope, fiber optics, fluid passage way, and the like. The outer cannula 101 is retracted back (with the push rod 105 held in place axially) to allow for deployment and fixation of the fixation ring 4 and the valve 20 secured thereto as shown in FIG. 13. The catheter 50′ is retracted further (FIG. 14) and out of the body.

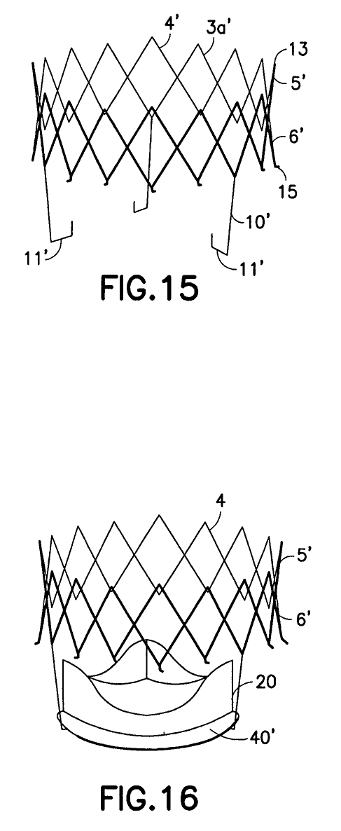

Turning now to FIG. 15, there is shown an alternate stent component 1′ for a stent-valve in accordance with the present invention. The stent 1′ is typically made from a laser machined shape memory metal or wire forms as described above. The stent 1′ contains a band of hexagonal shaped elements with adjacent elements sharing a common side, referred to as a fixation ring 4′. The fixation ring 4′ can also be comprised of diamond shaped or zig-zag shaped elements, etc. Each hexagonal element 3a′ is formed in a geometry such that both the upper apices 5′ and the lower apices 6′ extend radially outward from the central portion of the fixation ring 4′. Small barbs 13, 15 project from the apices 5′ and 6′, respectively, as shown. The purpose of the angle of the apices 5′, 6′ and the barbs 13, 15 is to contact the inner wall of a blood vessel in order to prevent the stent 1′from moving distally (or proximally) in the blood vessel; in other words, such apices and barbs aid in fixating the stent in place against the inner wall of the blood vessel.

A plurality (preferably, at least three) elements 10′ project generally downward (preferably from the bottom apices 6′ of the ring 4′) to feet 11′. The feet 11′ project radially inward and then upward as shown in FIG. 15. The feet 11′ support the non-collapsible valve element 20 as shown in FIG. 16. A seal 40′ is preferably disposed around the elements 10′ and the base of the valve element 20. The seal 40′ may be an annulus of foam, a multiplicity of strands, a rolled sewing cuff, or the like. The seal 40′ prevents blood from leaking around the valve element 20 once it is fixated. In addition, the seal 40′ can be made porous to allow tissue ingrowth and facilitate permanent fixation of the device. Further, for certain applications, such as for aortic valve replacement as discussed herein, the seal 40′ can also take the form of an annular wedge such that a wide potion of the wedge remains in the ventricle, while the remaining portion of the wedge lies in the aorta, much like a cork in a bottle.

The stent-valve device of FIG. 16 is preferably loaded into and deployed from a deployment catheter in a manner similar to that described above with respect to FIGS. 4-14. After the valve 20 is supported by the feet 11′, the fixation ring 4′ is compressed radially inwards (in a manner similar that shown in FIG. 4) and loaded into the catheter (e.g., into the nose cone 51 (FIG. 5) or in the outer cannula (FIG. 12)). The catheter is introduced into the body and located adjacent the intended deployment site. The catheter is manipulated to the deploy the fixation ring 4′ from the distal end of the catheter, where it expands and contacts the vessel wall for fixation of the ring 4′ and the valve 20 secured thereto. The catheter is then retracted out of the body. The apices and barbs of the fixation ring 4′ aid in fixating the stent-valve device 1′ in place against the inner wall of the blood vessel.

Advantageously, the prosthetic stent-valve devices described herein and the associated deployment mechanisms and surgical methods are minimally invasive and thus eliminate the multitude of sutures that are traditionally used to implant a heart valve. It also avoids total severing and re-suturing of the aorta which is standard practice for deploying prosthetic valves. By eliminating these complex procedures, the implantation time can be reduced significantly.

Although the above stent device is described as holding and deploying a non-collapsible prosthetic valve, it can be appreciated by those skilled in the art that the prosthetic valve, if designed to be compressed, can be made flexible and be compressed down and introduced through a small catheter. It is further appreciated by those skilled in the art that this device can be introduced percutaneously through a small hole in the iliac or femoral artery in the groin.

There have been described and illustrated herein several embodiments of a stent-valve assembly and a deployment catheter and surgical methods for use therewith. While particular embodiments of the invention have been described, it is not intended that the invention be limited thereto, as it is intended that the invention be as broad in scope as the art will allow and that the specification be read likewise. Thus, while particular geometries and configurations of the stent component have been disclosed, it will be appreciated that other geometries and configurations can be used as well. For example, the self-expanding fixation ring of the stent may be replaced by a fixation ring that is expanded through the use of an expandable balloon disposed inside the fixation ring. In addition, while particular configurations of the deployment catheter component have been disclosed, it will be understood that alternative configurations of the deployment catheter can be used. For example, instead of (or in conjunction with) a catheter housing or sheath that restrains the fixation ring, a suture can be used for this purpose. Once the fixation ring is located, the suture can be cut (or possibly pulled through) to release the fixation ring where it expands and fixates the stent-valve assembly in place. Such suture tension may be worthwhile as it keeps the valve from jumping which may happen when pushed from a catheter (commonly referred to as the “water melon seed” effect). Also, while particular applications have been disclosed for replacement of the aortic valve of the left ventricle of the heart, it can be readily adapted for use in the replacement of other heart valves (e.g., pulmonary valve). It will therefore be appreciated by those skilled in the art that yet other modifications could be made to the provided invention without deviating from its spirit and scope as claimed.

What is claimed is:

1. A stent-valve device comprising:
a non-collapsible valve component; and
a stent component having a first ring connected to a second ring, said first ring having a characteristic first diameter and a valve support which supports said valve component, and said second ring being contractible and expandable between a second diameter less than a third diameter, wherein said second diameter is less than said first diameter and said third diameter is greater than said first diameter.

2. A stent-valve device according to claim 1, wherein:
said valve support comprises a plurality of elements that extend downward to feet that project radially inward.

3. A stent-valve device according to claim 1, further comprising:
a seal disposed about said first ring.

4. A stent-valve device according to claim 1, further comprising:
a plurality of suspension elements that connect said first ring to said second ring.

5. A stent-valve device according to claim 1, wherein:
said second ring comprises a band of hexagonal elements having upper apices and lower apices extend radially outward in a manner that fixates said stent-valve device in place against an inner wall of a blood vessel.

6. A stent-valve device according to claim 1, wherein:
said second ring includes means for fixing said second ring in place against an inner wall of a blood vessel; and
said stent-valve device further comprises a plurality of suspension elements that connect said second ring to said first ring.

7. A stent-valve device according to claim 1, wherein:
said stent component is realized from at least one shape memory metal.

8. A stent-valve device according to claim 1, wherein:
said valve component comprises a substantially rigid annular base and a plurality of flexible leaflets that extend from said base.

9. A stent-valve device according to claim 8, wherein:
said valve component is one of mechanical valve prosthesis, a bio-prosthesis, and a polymer-based prosthesis.

10. A stent-valve device according to claim 9, wherein:
said valve component comprises a non-collapsible porcine valve.

11. An apparatus comprising:
a stent-valve device including
a valve component, and
a stent component having a first ring connected to a second ring, said first ring having a characteristic first diameter and a valve support which supports said valve component, and said second ring being contractible and expandable between a contracted state and an expanded state, said contracted state having a second diameter less than a third diameter of said expanded state, wherein said second diameter is less than said first diameter and said third diameter is greater than said first diameter; and
a deployment catheter including
a first housing that is adapted to store said second ring in its contracted state, and
means for moving said first housing axially to deploy said second ring from said first housing whereby it expands to its expanded state.

12. An apparatus according to claim 11, wherein:
said deployment catheter includes a first body member, operably coupled to said first housing, that is manipulated to effectuate axial movement of said first housing, and a restrictor member, operably disposed adjacent said second ring, that is adapted to limit axial movement of said second ring while said first body member is moved axially to deploy said second ring.

13. An apparatus according to claim 12, wherein:
said deployment catheter further comprises a second body member, operably coupled to said restrictor that is manipulated to effectuate axial movement of said first body member relative to said restrictor member.

14. An apparatus according to claim 13, wherein:
said second body member is concentric over said first body member.

15. An apparatus according to claim 13, wherein:
said valve component comprises an annular base and a plurality of flexible leaflets that extend from said base, and
said deployment catheter includes a second housing that is adapted to extend through said valve component.

16. An apparatus according to claim 11, wherein:
said valve component is non-collapsible.

17. An apparatus according to claim 16, wherein:
said valve component comprises a substantially rigid base and a plurality of leaflets that extend from said base.

18. An apparatus according to claim 16, wherein:
said valve component is one of mechanical valve prosthesis, a bio-prosthesis, and a polymer-based prosthesis.

19. An apparatus according to claim 15, wherein:
said deployment catheter further comprises a third body member adapted to effectuate axial movement of said second housing relative to said restrictor member and said first housing.

20. An apparatus according to claim 19, wherein:
said second body member is concentric over said first body member, and said third body member is concentric over both said first and second body members.

21. An apparatus according to claim 11, wherein:
said valve support comprises a plurality of elements that extend downward to feet that project radially inward.

22. An apparatus according to claim 11, wherein:
said stent-valve further comprises a seal disposed about said first ring.

23. An apparatus according to claim 11, wherein:
said stent component further comprises a plurality of suspension elements that connect said first ring to said second ring.

24. An apparatus according to claim 11, wherein:
said second ring comprises a band of hexagonal elements having upper apices and lower apices that extend radially outward in a manner that fixates said stent-valve device in place against an inner wall of a blood vessel.

25. An apparatus according to claim 11, wherein:
said second ring includes means for fixing said second ring in place against an inner wall of a blood vessel; and
said stent-valve further comprises a plurality of suspension elements that connect said second ring to said first ring.

26. A surgical method comprising:
providing an apparatus comprising a stent-valve device loaded into a deployment catheter,
said stent-valve device including a valve component and a stent component having a first ring connected to a second ring, said first ring having a characteristic first diameter and a valve support for supporting said valve component, and said second ring being contractible and expandable between a contracted state and an expanded state, said contracted state having a second diameter less than a third diameter of said expanded state, wherein said second diameter is less than said first diameter and said third diameter is greater than said first diameter, and
said deployment catheter including a first housing that stores said second ring in its contracted state, and means for effectuating axial movement of said first housing relative to said second ring;
inserting said apparatus into the body and guiding said deployment catheter to an intended deployment site;
axially moving said first housing relative to said second ring to cause said second ring to deploy from said first housing and automatically expand from its contracted state to its expanded state, whereby in its expanded state said second ring fixates said stent-valve device to an inner wall of a blood vessel at or near the intended deployment site; and
retracting said deployment catheter to remove it from the human body.

27. A surgical method according to claim 26, wherein:
said first housing is moved axially forward to cause said second ring to deploy from said first housing.

28. A surgical method according to claim 26, wherein:
said deployment catheter includes
a first body member adapted to effectuate axial movement of said first housing,
a restrictor member, operably disposed adjacent said second ring, that is adapted to limit axial movement of said second ring, and
a second body member adapted to effectuate axial movement of said restrictor member; and
wherein the method further comprises the step of manipulating said second body member to limit axial movement of said restrictor member while moving said first body member axially to deploy said second ring from said first housing.

29. A surgical method according to claim 28, wherein:
said valve component is non-collapsible, and
said deployment catheter includes
a second housing that extends through said valve component, and
a third body member adapted to effectuate axial movement of said second housing, and
wherein the method further comprises the step manipulating said third body member to retract said second housing from said valve component.

30. A surgical method according to claim 26, wherein:
said valve component is non-collapsible and preferably comprises a substantially rigid annular base and a plurality of flexible leaflets that extend from said base.

31. A surgical method according to claim 30, wherein:
said valve component is one of mechanical valve prosthesis, a bio-prosthesis, and a polymer-based prosthesis.

32. A surgical method according to claim 30, wherein:
said valve component comprises a non-collapsible porcine valve.

33. A surgical method according to claim 26, wherein:
said valve support comprises a plurality of elements that extend downward to feet that project radially inward.

34. A surgical method according to claim 26, wherein:
said stent-valve further comprises a seal disposed about said first ring.

35. A surgical method according to claim 26, wherein:
said stent component further comprises a plurality of suspension elements that connect said first ring to said second ring.

36. A surgical method according to claim 26, wherein:
said second ring comprises a band of hexagonal elements having upper apices and lower apices that extend radially outward in a manner that fixates said stent-valve device in place against an inner wall of a blood vessel.

37. A surgical method according to claim 26, wherein:
said second ring includes means for fixing said second ring in place against an inner wall of a blood vessel; and
said stent component further comprises a plurality of suspension elements that connect said second ring to said first ring.

38. A surgical method according to claim 26, wherein:
the intended deployment site is within the ascending aorta of the heart with said first ring positioned adjacent the left ventricle of the heart and said second ring positioned above the coronary arteries.

39. A surgical method according to claim 26, wherein:
the deployment catheter is introduced below the intended deployment site.

40. A surgical method according to claim 26, wherein:
the deployment catheter is introduced above the intended deployment site.
